(19) United States
(12) Patent Application Publication
(10) Pub. No.: US 2015/0328430 A1
(43) Pub. Date: Nov. 19, 2015

(54) RAPID, PRECISE, NITRIC OXIDE ANALYSIS AND TITRATION APPARATUS AND METHOD

(71) Applicant: SYK TECHNOLOGIES, LLC, Newport Beach, CA (US)

(72) Inventors: J. W. Randolph Miller, Orem, UT (US); Ross A. Wille, Orem, UT (US)

(21) Appl. No.: 14/810,303

(22) Filed: Jul. 27, 2015

Related U.S. Application Data
(63) Continuation-in-part of application No. 14/194,977, filed on Mar. 3, 2014.
Application No. 14/194,977 is a continuation of application No. 13/197,695, filed on Aug. 3, 2011, now Pat. No. 8,685,467.
(60) Provisional application No. 62/138,856, filed on Mar. 26, 2015.
(60) Provisional application No. 61/370,214, filed on Aug. 3, 2010.

(51) Int. Cl. A61M 16/14; A61M 16/20; A61M 16/00; A61K 33/00; A61K 47/02

(52) U.S. Cl. CPC A61M 16/14; A61K 33/00; A61K 47/02; A61M 16/0003; A61M 16/0057; A61M 16/208; A61M 2202/0275; A61M 2205/3334; A61M 2205/50

(57) ABSTRACT

An apparatus for controlled delivery of nitric oxide uses a processor receiving inputs from a detector to control upstream introduction of the nitric oxide into breathing air. Improved accuracy and response speed are achieved by automatic control over a needle valve metering nitric oxide into breathing air. Also, a diverter vectoring a sample of the mixed air and nitric oxide toward a face of the detector reduces the diffusion boundary layer resulting in greater precision and speed of response.

### RELATED APPLICATIONS

This application claims the benefit of U.S. Provisional Patent Application Ser. No. 62/138,856, filed Mar. 26, 2015, entitled RAPID, PRECISE, NITRIC OXIDE ANALYSIS AND TITRATION APPARATUS AND METHOD, and is a continuation-in-part of U.S. patent application Ser. No. 14/194,977, filed Mar. 3, 2014, entitled NITRIC OXIDE GENERATION, DILUTION, AND TOPICAL APPLICATION APPARATUS AND METHOD, which is a continuation of U.S. patent application Ser. No. 13/197,695, filed Aug. 3, 2011, entitled NITRIC OXIDE GENERATION, DILUTION, AND TOPICAL APPLICATION APPARATUS AND METHOD, issued as U.S. Pat. No. 8,685,467 on Apr. 1, 2014, which claims the benefit of U.S. Provisional Patent Application No. 61/370,214, filed Aug. 3, 2010, entitled NITRIC OXIDE GENERATOR AND DILUTION APPARATUS AND METHOD; all of which are hereby incorporated by reference in their entirety.

### BACKGROUND

1. the Field of the Invention

This invention relates generally to measurement and control, and, more specifically, to apparatus and methods for analyzing and controlling delivery of nitric oxide over a comparatively wide range of dosage rates.

2. Background

The discovery of certain nitric oxide effects in live tissue garnered a Nobel prize. Much of the work in determining the mechanisms for implementing, and the effects of, nitric oxide administration are reported in literature. In its application however, introduction of nitric oxide to the human body has traditionally been extremely expensive. The therapies, compositions, preparations, hardware, and controls are sufficiently complex, large, and expensive to inhibit more widespread use of such therapies.

What is needed is a comparatively simple, easily controlled, and consequently inexpensive mechanism for introducing nitric oxide in a variable concentration. Also, needed is a simple introduction method for providing nitric oxide suitable for inhaling. Also, needed is a simple method for topical application of a nitric oxide therapy. Precisely and responsively over a broader range from well below 100 parts per million (ppm) (even down to 10 ppm), in the infant dosing range, up to about 600 ppm for adult dosing, and over 1000 ppm for topical and other applications control and administration would be a great benefit from simplicity and reduction in size.

It would be an advance in the art to provide a system suitable for administration of nitric oxide gas at precise, stable, yet variable concentrations whether or not from bottled gas.

### BRIEF SUMMARY OF THE INVENTION

In accordance with the foregoing, certain embodiments of apparatus and methods in accordance with the invention provide a reactor system that produces nitric oxide and regulates the flow and concentration of nitric oxide delivered. Nitric oxide may thus be introduced into the breathing air of a subject in a controlled manner. Nitric oxide amounts may be engineered to deliver a therapeutically effective amount on the order of single digits to the comparatively low hundreds (e.g., 100-500) of parts per million, or up to thousands of parts per million.

For example, sufficient nitric oxide may be presented through nasal inhalation to provide approximately five thousand parts per million in breathing air. This may be diluted due to additional bypass breathing, through nasal inhalation, or through oral inhalation.

One embodiment of an apparatus and method in accordance with the present invention may rely on a small reactor and a system of filters and pumps configured to provide a constant, regulated flow of nitric oxide. Other embodiments may provide an automated feedback system that monitors, controls, and adjusts the concentration of nitric oxide delivered.

Reactive compounds may be appropriately combined dry or in liquid form. Reactants may include potassium nitrite, sodium nitrite or the like. The reaction may begin upon introduction of heat. Heat may be initiated by liquid transport material to support ionic or other chemical reaction in a heat device.

An apparatus and method in accordance with the invention may include an insulating structure, shaped in a convenient, compact, efficient configuration such as a rectangular box, a cylindrical container, or the like. The insulating container may be sealed either inside or out with a containment vessel to prevent leakage of liquids therefrom. Such a system may not need to be constructed to sustain nor contain pressure. However, in certain embodiments, the reactor may need to be constructed to sustain and contain pressure.

In certain embodiments, chemical heaters may include metals finely divided to readily react with oxygen or solid oxidizers. Inside the containment vessel may be positioned heating elements such as those commercially available as chemical heaters. Various other chemical compositions of modest reactivity may be used to generate heat readily without the need for a flame, electrical power, or the like.

Above the heating element or heater within the containment vessel may be located a reactor. The reactor may preferably contain a chemically stable composition for generating nitric oxide. Such compositions, along with their formulation techniques, shapes, processes, and the like are disclosed in U.S. patent application Ser. No. 11/751,523, U.S. patent application Ser. No. 12/361,123, U.S. patent application Ser. No. 12/361,151, U.S. patent application Ser. No. 12/410,442, U.S. patent application Ser. No. 12/419,123, and U.S. Pat. No. 7,220,393, all incorporated herein by reference in their entireties as to all that they teach.

The reactor may include any composition suitable for generating nitric oxide by the activation available from heat. The reactor may be substantially sealed except for an inlet, such as a tubular member secured thereto to seal a path for entry of filtered air into the reactor, and an outlet, such as a tubular member secured thereto to seal a path for exit of nitric oxide from the reactor. The reactor may also include a structure to dissipate heat away from the reaction and facilitate the complete use of the reactants in the reactor.

In certain embodiments, a system of filters and pumps introduces air into the reactor and then conducts a controlled flow of nitric oxide out of the reactor. Accordingly, a system may include filters and pumps to introduce air into the reactor, control production of nitric oxide in the reactor, and conduct nitric oxide out of the reactor. The system may include devices controlling the pumps and the flow of nitric oxide.

Ultimately, an apparatus in accordance with the invention may include a cover through which an outlet penetrates from the reactor in order to connect to a cannula. This has been done effectively. The cover may also vent steam generated by the heaters in the presence of the water typically used to activate such heaters.

The system may be configured for continual use by replenishing the reactants and replacing other components as needed. Alternatively, the system may be completely wrapped in a pre-packaged assembly. In one embodiment, a heat-shrinkable wrapping material may be used to seal the outer container of an apparatus in accordance with the invention. Thus, this system may be rendered tamper-proof, while also being maintained in integral condition throughout its distribution, storage, and use.

In accordance with the foregoing, certain embodiments of an apparatus and method in accordance with the invention provide a topical medium that produces nitric oxide and provides a therapeutic concentration of nitric oxide delivered to a surface. Nitric oxide may thus be introduced to the skin, or a wound, of a subject in a controlled manner. Nitric oxide amounts may be engineered to deliver a therapeutically effective amount on the order of from comparatively low hundreds (e.g., 100-500) of parts per million, up to thousands of parts per million. For example, sufficient nitric oxide may be presented through topical application to provide approximately five hundred parts per million to the surface of a subject's skin.

One embodiment of an apparatus and method in accordance with the present invention may rely on equal amounts of a nitrite medium and an acidified medium formulated to provide a burst of nitric oxide, as well as a continuous amount of nitric oxide over a period of time. One embodiment of an apparatus and method in accordance with the present invention may provide a therapeutically effective amount of nitric oxide from a gel medium, which provides a therapeutically effective dose of nitric oxide over a relatively shorter length of time, from approximately thirty minutes up to about 3 hours.

One embodiment of an apparatus and method in accordance with the present invention may provide a therapeutically effective amount of nitric oxide from a lotion medium, which provides a therapeutically effective dose of nitric oxide over a relatively longer length of time, from about one hour up to about 6 hours. Reactants may include potassium nitrite, sodium nitrite or the like. The reaction may begin upon combination of the nitrite medium and the acidified medium.

An apparatus and method in accordance with the invention may be used for a variety of purposes, including without limitation, disinfecting and cleaning surfaces, increasing localized circulation, facilitating healing and growth, dispersing biofilms, and providing analgesic benefits.

### BRIEF DESCRIPTION OF THE DRAWINGS

The foregoing features of the present invention will become more fully apparent from the following description, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a schematic view of one embodiment of an apparatus in accordance with the invention to generate nitric oxide and control the flow and concentration of nitric oxide delivered;

FIG. 2 is a perspective view of a containment vessel, or cannister;

FIG. 3 is a top perspective view of an open containment vessel, or cannister;

FIG. 4A is a cross-sectional view of a containment vessel, or cannister;

FIG. 4B is a close-up view of the center, bottom of the cross-sectional view of the containment vessel to more clearly show the heat cartridge sleeve of the containment vessel;

FIG. 5 is a schematic view of an automated feedback system that can monitor and adjust the flow or concentration of nitric oxide provided to a ventilator system;

FIG. 6 is a schematic of a possible combination a nitrite medium and an acidified medium for production of a topical medium for topical application of nitric oxide therapy;

FIG. 7 is a schematic block diagram of a computer system for implementing a programmed control process for a system in accordance with the invention;

FIG. 8 is a schematic block diagram of a hardware suite implementing one embodiment of an analysis and control system for administering nitric oxide gas therapy to a subject;

FIG. 9 is a schematic block diagram of one alternative embodiment thereof;

FIG. 10 is a side elevation, cross-sectional, schematic view of a fluid boundary layer near a sensor;

FIG. 11 is a side elevation, cross-sectional view thereof showing vectored flows to decrease thickness of the boundary layer;

FIG. 12 is a side elevation, cross-sectional view of one embodiment of a needle valve for use in an apparatus in accordance with the invention; and

FIG. 13 is a chart showing examples of monotonic approaches to adjustment of independent and dependent variables by a metering valve controller in accordance with the invention.

### DETAILED DESCRIPTION OF THE PREFERRED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings.

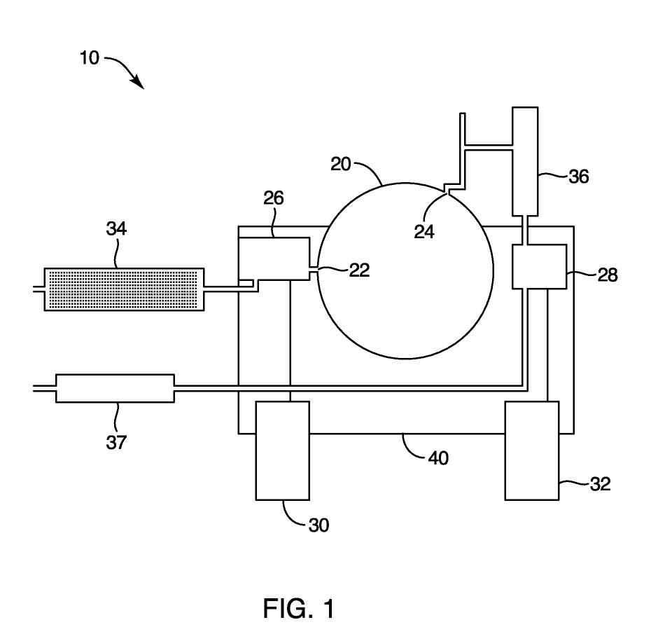

Referring to FIG. 1, a nitric oxide generator 10 may include a first pump 26 that draws air through an activated carbon filter 34 and pressurizes the reaction chamber 20, or reactor 20. The pump 26 provides filtered air for dilution with the nitric oxide to be generated. The pump 26 pumps air into the reactor 20 and pressurizes the reactor 20. Any device suitable for pumping air into and pressurizing the reactor 20 may be utilized.

The pump 26 may be controlled by a potentiometer 30, or the like. Using a potentiometer 30 allows the voltage to the pump 26 to be varied according to the desires of the user. The potentiometer 30 may include circuit boards that control the speed of the pump 26. Also, pump controls that control and measure the amperage to the pumps as opposed to the voltage may also be utilized when measuring the amperage is simpler, easier, or more useful for controlling the pump speed and power. Any device suitable for controlling the pump may be utilized.

The activated carbon filter 34 filters out oxygen and moisture from the inlet air. Again, any suitable device may used to filter the inlet air appropriately. In another embodiment, the first pump 26 may pump air through the activated carbon filter 34 and then into the reaction chamber 20.

A reaction chamber 20 provides a suitable container for the reaction that produces the nitric oxide. The reaction chamber 20 can be of any suitable size or shape. The various configurations for a suitable reaction chamber 20, as well as the compounds and components used in the reaction, are described elsewhere hereinafter. However, compactness for portability and home use may be valuable.

A vent, or outlet 24, in the reaction chamber 20 allows air and nitric oxide to be drawn out of the reaction chamber 20. The outlet 24 may be configured to release excess pressure in the reaction chamber 20 by allowing air and nitric oxide to escape the system to the atmosphere. The outlet 24 may also be configured to direct the air and nitric oxide from the reactor to a first calcium hydroxide filter 36. The outlet 24 allows venting of the flow through the reactor and helps make sure the proper flow goes through the orifice. The system may provide means for applying a constant flow to the orifice and then venting overboard any remaining or excess flow of nitric oxide.

A second pump 28 draws air and nitric oxide through the first calcium hydroxide filter 36 away from the reaction chamber 20 for use in any type of nitric oxide therapy. The pump 28 further dilutes the nitric oxide with filtered air. The pump 28 may be controlled by a second potentiometer 32, or the like. Using a potentiometer allows the voltage to the pump 28 to be varied according to the desires of the user. The potentiometer may include circuit boards that control the speed of the pump. Also, pump controls that control and measure the amperage delivered to the pumps as opposed to controlling the voltage as described above. Any device suitable for controlling the pump may be utilized. The calcium hydroxide filter 36 absorbs or otherwise filters out moisture and scavenges nitrogen dioxide (NO₂) from the outlet air. Again, any other suitable device may used to filter or otherwise clean the outlet air appropriately.

A line from the second pump 28 is used to conduct nitric oxide away from the reactor 20 and deliver the nitric oxide for use in various nitric oxide therapies. An orifice at one end of this line is used to restrict and control the flow of nitric oxide. The nitric oxide travels from the second pump 28 through this line, through the orifice, and through a second calcium hydroxide filter 37.

This line from the second pump to the orifice may be a ⅛ inch stainless steel line that carries gas and resists heat and corrosion. Any line used in this system may be a stainless steel line that carries gas and resists heat and corrosion, or any suitable device or material that can conduct the flow of gas in an acceptable manner. Also, any line in the system may be of silicone tubing that is resistant to heat, alcohol, and castor oil. Moreover, any line in the system may be composed of any material that is suitable for the intended purpose, including without limitation, stainless steel, medical grade silicone, plastic, or the like.

The orifice used to restrict and control the flow of nitric oxide may have an aperture from about 2 to about 10 mils, and typically about 0.004 inches in diameter. Any suitable aperture that will restrict and control (e.g., effectively meter) the flow of nitric oxide at a desired level. For example, orifice or aperture may typically be of any size from approximately 0.003 inches to 0.009 inches in diameter.

Finally, the second calcium hydroxide filter 37 removes any remaining moisture and nitrogen dioxide from the gas exiting the reactor 10. After passing through this second calcium hydroxide filter 37, the nitric oxide is ready for use with any variety of nitric oxide therapies. Also, the nitric oxide may be diluted with the air delivered to the patient.

The nitric oxide reactor 10 may include a cover 40 to contain the components of the reactor. The cover 40 may be any suitable shape and material and may be designed to allow access to the components of the reactor 10. The cover 40 may also be designed to enclose a reactor 10 intended for a single use by a patient. Such a single use reactor may be discarded or returned to an appropriate facility for recycling the reactor and its components.

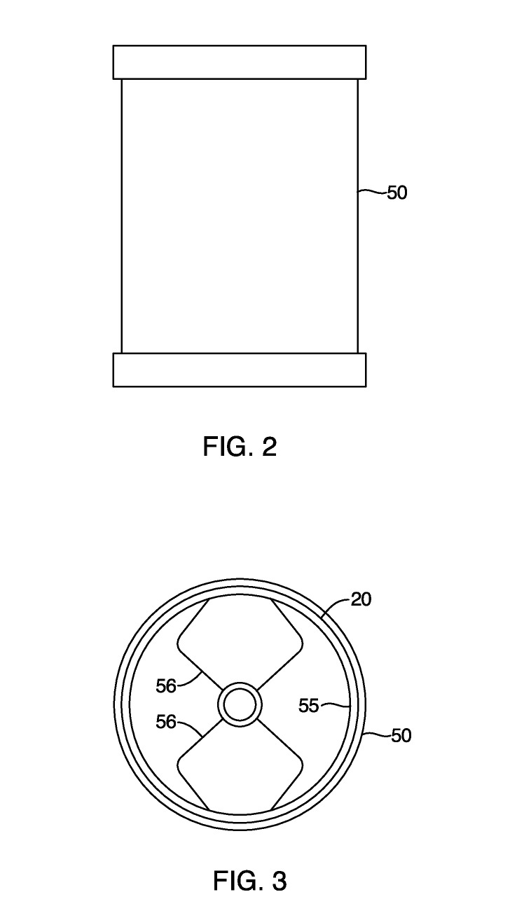

Referring to FIG. 2, in one embodiment the reaction chamber 20 may be contained within a containment vessel 50, or cannister 50. The top of the containment vessel 50 may be configured to be secured, such as by being screwed on to the containment vessel 50, to close or seal the reaction chamber, or unscrewed to allow access to the reaction chamber. The containment vessel may be heated by any suitable heating element. The containment vessel may be of any suitable configuration and may be made of any suitable material, such as stainless steel.

Referring to FIG. 3, in one embodiment the reaction chamber 20 may include fins 56, fin-like structures 56, in contact with the heating element 58 of the reaction chamber 20 and the outside wall 55 of the reaction chamber 20. These fins 56 dissipate the heat of the reaction and facilitate a complete nitric oxide reaction and use of all the reactants. These fins 56 may be composed of any serviceable heat transfer material that will not interfere with the reaction in the reactor and will stay in contact with the heating element 58 and the outside wall 55 of the reactor. Fins 56 may be designed to provide a constant contact force between the heater in the reactor and the wall of the reaction chamber 20. Fins 56 may be intimately bonded or may be described as “spring-loaded” fins in forced contact with the walls of the reaction chamber 20. The fins 56 are especially helpful when the reactants for the nitric oxide reaction include a powder, in which conductive heat transfer through the reactants is comparatively poor.

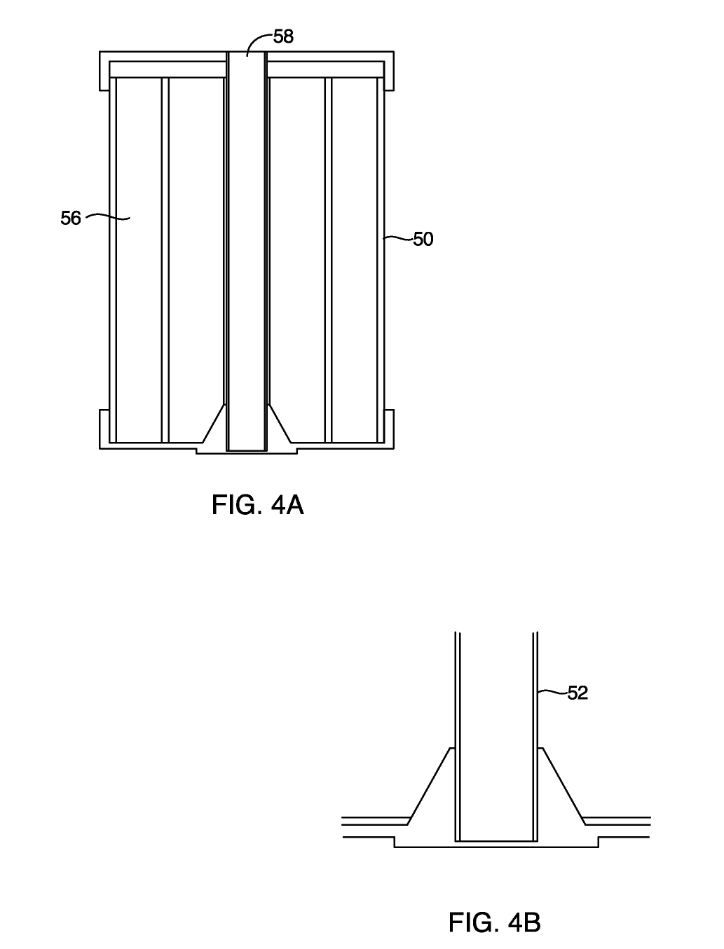

Referring to FIG. 4A, in one embodiment the reaction chamber 20 may be configured to allow for a heating element 58, or cartridge, extending axially along the containment vessel 50. Referring to FIG. 4B, the containment vessel 50 may also include a heat cartridge sleeve 52 to accommodate the heating element 58, or cartridge.

In one embodiment the formulation for the reactants may include the following: approximately 2.3 kg of calcined chromium oxide (Cr₂O₃) or approximately 51% of the granulation, approximately 1.6 kg of sodium nitrite (NaNO₂) or approximately 34.7% of the granulation, and approximately 0.65 kg of sodium nitrate (NaNO₃) or approximately 14.4% of the granulation. These amounts can be adjusted to provide an optimal production of nitric oxide. Generally, the amounts for the respective components may be adjusted plus or minus 10% of the granulation.

Calcined products are best stored under vacuum. The components are best ground to produce a loose granulation passing through a 5 micron screen. Each of the components should go through a double grind separately. All the components should be ground together a third time. The resulting granulation should be stored under nitrogen (N₂) or under vacuum at a comparatively cooler temperature than room temperature (lower is better) and in low light or no light conditions.

In one embodiment, the concentration of nitric oxide delivered can be varied anywhere from 0 ppm to one million ppm. Principally, the nitric oxide may be diluted with outside air. However, the system may be configured such that the nitric oxide can be diluted with any designated gas. Excess gas or nitric oxide can be vented to the atmosphere. The concentration can be adjusted rapidly in order to respond to the protocols and parameters of a variety of nitric oxide therapies.

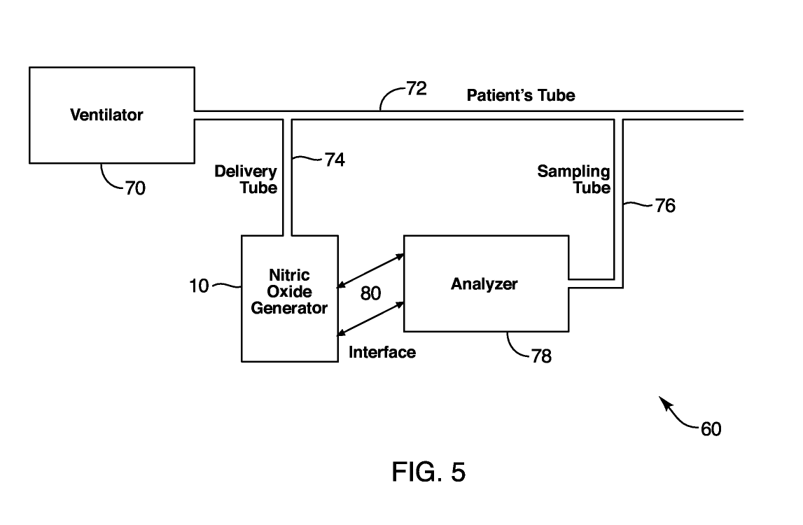

Referring to FIG. 5, in one embodiment, an integrated system 60 may be utilized to control and adjust the delivery of nitric oxide. Such a system may sample or measure the concentration of nitric oxide delivered to a user and then automatically adjust the amount of nitric oxide delivered to the air flow of the user. For example and not by way of limitation, a nitric oxide therapy may be delivered to a patient using a ventilator 70 with a breathing tube 72. After the nitric oxide is delivered to the air flow in the breathing tube through a delivery tube 74, a sample is taken through a sampling tube 76, or the air flow is measured, to determine the concentration of nitric oxide. Any device suitable for analyzing 78 or measuring the concentration of nitric oxide may be used. After a determination is made with regard to the concentration of nitric oxide, the amount of nitric oxide delivered to the air flow in the breathing tube can be adjusted by adjusting the controls of the nitric oxide dilution apparatus, such as adjusting the speed of the pumps or a bypass air inlet in the apparatus.

In one embodiment, an integrated system 60 includes a feedback loop. Measuring, adjusting, and controlling the concentration of nitric oxide may be monitored and controlled by an interface 80 device.

Again referring to FIG. 1, one embodiment of an apparatus and method in accordance with the invention may rely on a series of process steps constituting a method or process. For example, providing a pump may involve any one or more of the required tasks of identifying materials and determining the structural and mechanical characteristics for such a pump. Accordingly, providing a pump may involve design, engineering, manufacture and acquisition of such a device. Similarly, providing a potentiometer to control a pump by varying the voltage or current to the pump may involve identifying materials and determining the structural and mechanical characteristics for such a potentiometer. Accordingly, providing a potentiometer may involve design, engineering, manufacture, and acquisition of such a device.

Providing an activated carbon filter may involve identifying materials, selecting a shape, selecting a cross-sectional profile and active area, and determining the structural and mechanical characteristics for such a filter. Similarly, providing a calcium hydroxide filter may involve identifying materials, selecting a shape, selecting a cross-sectional profile, evaluating an active area, and determining the structural and mechanical characteristics for such a filter. Accordingly, providing any type of filter may involve design, engineering, manufacture and acquisition of such a device.

Providing a reactor may involve selection of materials, selection of a profile and of cross-sectional area, engineering, design, fabrication, acquisition, purchase, or the like of a reactor in accordance with the discussion hereinabove.

Providing reactants may include selection of reacting species, selecting a configuration, such as granules, powder, liquid, gel, a solution, multiple components to be mixed, or the like. Likewise, the particular configuration of a solidous configuration of reactants may involve selecting a sieve size for the particles. This size can affect surface area available to react, heat penetration distances, and times controlling overall chemical reaction rates. Thus, selecting or otherwise providing reactants for the reactor may involve consideration of any or all aspects of chemistry, reaction kinetics, engineering, design, fabrication, purchase or other acquisition, delivery, assembly, or the like.

Assembling the apparatus may also include the disposition of reactants within various locations within a reactor, system, or the like as discussed hereinabove. Activating the reactants in the reactor may involve, either adding a liquid, mixing the reactant components together, dispersing individual reactants in respective solutes to provide solutions for mixing, adding a liquid transport carrier to dry ingredients in order to initiate exchange between reactants, heating the reactants, a combination thereof, or the like.

Likewise, activation of the reactants may also involve opening valves, opening seals, rupturing or otherwise compromising seals as described hereinabove, or otherwise moving or manipulating reactants with or without carriers in order to place them in chemical and transport contact with one another.

In certain embodiments, nitric oxide may be separated from the reactants themselves. For example, the concept of a molecular sieve as one mechanism to separate nitric oxide form other reactants and from other species of nitrogen compounds is possible. In other embodiments, pumps, vacuum devices, or the like may also tend to separate nitric oxide. Accordingly, in certain embodiments, a suitably sized pump may actually be connected to the reactor in order to draw nitric oxide away from other species of reactants or reacted outputs.

Conducting therapy using nitric oxide may involve a number of steps associated with delivery and monitoring of nitric oxide. For example, in certain embodiments, conducting therapy may involve activating a reactor or the contents thereof.

Monitoring may involve adding gauges or meters, taking samples, or the like in order to verify that the delivery of nitric oxide from the reactor to the user does meet the therapeutically designed maximum and minimum threshold requirements specified by a medical professional.

Ultimately, after the expiration of an appropriate time specified, or the exhaustion of a content of a reactor, a therapy session may be considered completed. Accordingly, the apparatus may be removed from use, discarded, or the like. Accordingly, the removal or discarding of the apparatus may be by parts, or by the entirety.

It is contemplated that the reactor may typically be a single dose reactor but need not be limited to such. Multiple-dose or reusable reactors may also be used. For example, the reactor may actually contain a cartridge placed within the wall. The internal structure of the cartridge may be ruptured in the appropriate seal locations, such as by a blade puncturing the seals by a mechanism on, in, or otherwise associated with the main containment vessel or wall, and thus activated. Accordingly, the reactor may be reused by simply replacing the cartridge of materials containing the reactant volumes.

A patient may also obtain the benefits of nitric oxide therapy by utilizing a topical application that generates nitric oxide. The nitric oxide may affect the surface to which the topical application is applied, and may be absorbed by a surface such as skin.

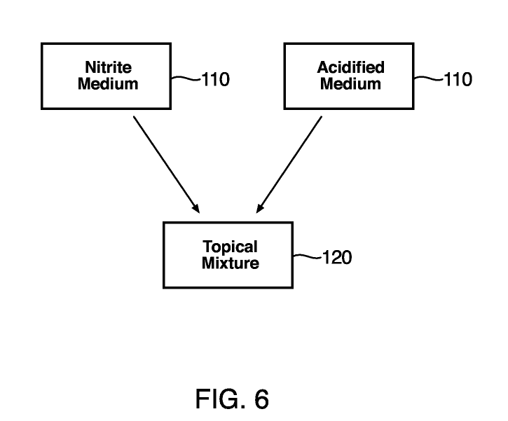

Referring to FIG. 6, two individual, separate, component media are provided. The first medium is a nitrite medium 100 and generally provides the nitrite reactants in some suitable form described herein above, such as sodium nitrite, potassium nitrite, or the like. The second medium is an acidified medium 110 and generally provides at least one acidic reactant in some suitable form, such as citric acid, lactic acid, ascorbic acid, or the like. Reaction rate and pH control are best achieved by using a mixture of multiple food-grade acids. When approximately equal amounts of the two individual components (media) are combined into a topical mixture 120, a reaction is initiated that produces nitric oxide.

Two containers may be provided, each container is capable of dispensing a suitable amount of a given medium (one of the two to be mixed). The containers may be identical in structure and composition, but need not necessarily be so. The containers may dispense the medium by a pump action, such as is common with lotions and soaps. The containers may dispense the medium by a squeezing or shaking action, such as is common with viscous or thixotropic shampoos, condiments, colloidal suspensions, gels, and other compositions.

The medium may be any suitable medium for containing and dispensing the reactants, for example, the medium may be a gel or a lotion. A gel may be obtained by including a water-soluble polymer, such as methyl cellulose available as Methocel™, in a suitable solution. A lotion used to suspend the reactants for a nitrite lotion medium and an acidified lotion medium may be selected such as the Jergens® brand hand and body lotion. For best results, the media holding a matched pair of reactants should be essentially the same. The chemical characteristics of the media may not be strictly identical, but the physical compositions should be essentially the same so as to mix readily and not inhibit the reaction.

For example, a nitrite gel medium may have a slightly acidic to neutral pH while an acidified gel medium may have a more acidic pH than the corresponding nitrite gel medium. Using a nitrite gel medium with an acidified lotion medium may not provide optimal results. Using different media may not provide the best rates for desired results, but would probably not be dangerous.

Generally, a topical application of nitric oxide may be provided by mixing equal amounts of a nitrite medium 100 and an acidified medium 110. The mixture 120 is then applied to the intended surface. The mixture 120 may be applied to a person's skin, or even an open wound.

The mixture 120 provides nitric oxide to the intended surface. As the nitrite medium 100 is mixed with the acidified medium 110, the reduction of nitrite by the acid(s) leads to the release of nitric oxide. The exposure to nitric oxide may serve a variety of purposes.

A topical mixture 120 that produces nitric oxide may be used for antimicrobial, antifungal, or similar cleaning purposes. Infectious diseases are caused by pathogens such as bacteria, viruses, and fungi. Antibacterial soaps can kill some bacteria, but not necessarily all bacteria. A topical mixture as described has been shown to kill as many as, and more, bacteria compared to commercially available antibacterial soaps or hospital-based instant hand antiseptics.

A topical mixture 120 that produces nitric oxide may be used for localized analgesic purposes. The analgesic effect nitric oxide may be provided via topical application.

A topical mixture 120 that produces nitric oxide may be used for anti-inflammatory purposes. A topical mixture that produces nitric oxide may also be used to disperse a biofilm. Biofilms are colonies of dissimilar organisms that seem to join symbiotically to resist attack from antibiotics. Nitric oxide signals a biofilm to disperse so antibiotics can penetrate the biofilm. It is also believed that nitric oxide interferes with the uptake of iron.

A topical mixture 120 that produces nitric oxide may be used to help heal various kinds of wounds. Tests have been performed wherein a topical mixture that produces nitric oxide as described herein is applied regularly to an open wound that is generally resistant to healing. The wound was seen to show significant healing within a few weeks.

For example, a person in Canada had poor circulation and unresponsive diabetic ulcers on the person's feet. The person was immobilized and in a wheel chair, and had been scheduled for amputation to remove the person's foot about a month after this experiment began. A topical mixture 120 that produces nitric oxide was applied to the diabetic ulcers once a day. The person soaked the effected foot in a footbath solution that produces nitric oxide for approximately twenty minutes once every four days. Within two weeks the person was able to walk and go out in public. Within 4-6 weeks, the person was mobile and had achieved a substantially complete recovery. Meanwhile, the scheduled amputation was cancelled.

It was shown that a topical mixture that produces nitric oxide will kill squamous cells, pre-cancerous cells, if the concentration of nitric oxide is high enough. Tests intending to show that a topical mixture that produces nitric oxide would grow hair based in part on the increase of blood flow that accompanies application of nitric oxide actually showed that nitric oxide in as high doses provided as described herein above did kill squamous cells.

The nitrite medium 100 may be formulated in any suitable medium and the concentration of reactants can be adjusted as desired as long as the intended reaction and sufficient concentrations of nitric oxide is obtained. For example, a suitable tank may be charged with distilled/deionized water (94.94% w/w) at room temperature (20°-25° C.). Sodium nitrite (3.00% w/w) and Kathon CG (0.05% w/w) may be dissolved in the water. Methocel™ (HPMC, cold dispersable; 1.75% w/w) may be stirred into the water until no lumps are present. Sodium hydroxide (10N to approximately pH 8; 0.09% w/w) may be rapidly stirred into the water to thicken, and care should be taken to avoid trapping air bubbles that can occur as a result of higher shear mixing.

EDTA, Na4 salt (0.10% w/w) may be stirred into the water until dissolved. Citric acid (crystalline; 0.08% w/w) may be added to adjust the mixture to a pH of 6.0. Small quantities of sodium hydroxide may be used to adjust the pH as needed. The individual percentages may be adjusted as desired for the best results.

The acidified medium 110 may be formulated in any suitable carrier and the concentration of the reactants can be adjusted as desired as long as the intended reaction and sufficient concentrations of nitric oxide are obtained. For example, a suitable tank may be charged with distilled/deionized water (89.02% w/w) at room temperature (20°-25° C.). Kathon CG (0.05% w/w) may be dissolved in the water. Methocel™ (HPMC, cold dispersable; 1.75% w/w) may be stirred into the water until no lumps are present. Sodium hydroxide (10N to approximately pH 8; 0.09% w/w) may be rapidly stirred into the water to thicken, and care should be taken to avoid trapping air bubbles that can occur as a result of higher shear mixing.

EDTA, Na4 salt (0.10% w/w) may be stirred into the water until dissolved. Stirring may continue until the Methocel™ is completely hydrated. Lactic acid (85% liquid solution; 3.00% w/w) and ascorbic acid (USP, crystalline; 3.00% w/w) may be stirred in until completely dissolved. Citric acid (crystalline; 3.00% w/w) may be added to adjust the mixture to a pH of 6.0. Small quantities of sodium hydroxide may be used to adjust the pH as needed. The individual percentages may be adjusted as desired for the best results.

The use of at least two acids in producing the acidified medium 110 may improve the shelf life of the acidified medium 110. Generally maintaining a pH of from about 3 to about 5 or above (so long as not too caustic for skin) has been found very useful in maintaining the shelf life of the product.

A topical mixture 120 that produces nitric oxide has been shown to be effective in cleaning and disinfecting hands. For example, three sets of volunteers, with approximately 26 people in each set, participated in a test to determine the effectiveness of nitric oxide as a cleaning and disinfecting agent. The right and left hands of each person in each set of volunteers were swabbed with cotton-tipped applicators prior to any type of washing. The applicators were plated onto nutrient blood agar petri dishes using the three corner dilution method.

Each set of volunteers washed their hands using separate soaps for washing. The first set of volunteers washed their hands for thirty (30) seconds using a topical mixture 120 of equal parts of nitrite gel medium and acidified gel medium as described herein above. The second set of volunteers washed their hands for thirty (30) seconds using a commercial anti-bacterial agent Avagard™D. The third set of volunteers washed their hands for fifteen (15) seconds using Dial™ Complete Foaming Hand Wash, and then rinsed for fifteen (15) seconds and dried.

The right and left hands of each person in each set of volunteers were swabbed again with cotton-tipped applicators after washing. The applicators were plated onto nutrient blood agar petri dishes using the three corner dilution method. All the blood agar petri dishes were incubated for forty-eight (48) hours at 35° C. The results were tabulated based on a grading scale of bacteria colonization. The testing showed that a topical mixture that produces nitric oxide reduced the relative bacterial content by approximately 62%. Avagard™D reduced the relative bacterial content by approximately 75%. Dial™ Complete Foaming Hand Wash reduced the relative bacterial content by approximately 33%. Thus, a topical mixture that produces nitric oxide was found to be approximately twice as effective and cleaning and disinfecting hands than Dial™ Complete Foaming Hand Wash and almost as effective as Avagard™D.

It has been determined that the dose required to kill bacteria on a surface, such as a person's skin, is at least approximately 320 ppm of nitric oxide. A topical gel mixture of approximately three (3) grams of nitrite gel medium and approximately three (3) grams of acidified gel medium that produces nitric oxide has been shown to deliver approximately 840 ppm of nitric oxide. Similarly, a topical gel mixture of approximately three (3) grams of nitrite lotion medium and approximately three (3) grams of acidified lotion medium that produces nitric oxide has been shown to deliver approximately 450 ppm of nitric oxide.

Measurement, control, and stability of flows of nitric oxide are another matter. Timely and precise control is not available. Closed loop control is not used in therapy. Coarse (imprecise) control and no automatic feed back are the norm. Speed and precision over a wide range of flow rates is now available.

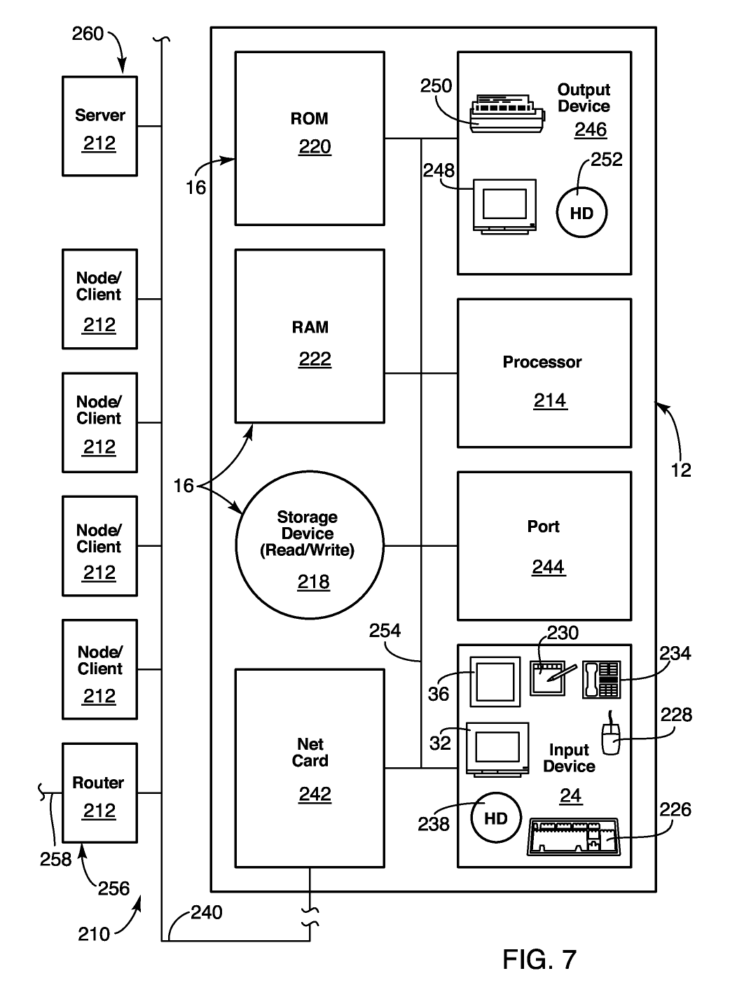

Referring to FIG. 7, a computer apparatus 210 or system 210 for implementing various aspects of the present invention may include one or more nodes 212 (e.g., client 212, computer 212). Such nodes 212 may contain a processor 214 or CPU 214. The CPU 214 may be operably connected to a memory device 216. A memory device 216 may include one or more devices such as a hard drive 218 or other non-volatile storage device 218, a read-only memory 220 (ROM 220), and a random access (and usually volatile) memory 222 (RAM 222 or operational memory 222). Such components 214, 216, 218, 220, 222 may exist in a single node 212 or may exist in multiple nodes 212 remote from one another.

In selected embodiments, the computer apparatus 210 may include an input device 224 for receiving inputs from a user or from another device. Input devices 224 may include one or more physical embodiments. For example, a keyboard 226 may be used for interaction with the user, as may a mouse 228 or stylus pad 230. A touch screen 232, a telephone 234, or simply a telecommunications line 234, may be used for communication with other devices, with a user, or the like. Similarly, a scanner 236 may be used to receive graphical inputs, which may or may not be translated to other formats. A hard drive 238 or other memory device 238 may be used as an input device whether resident within the particular node 212 or some other node 212 connected by a network 240. In selected embodiments, a network card 242 (interface card) or port 244 may be provided within a node 212 to facilitate communication through such a network 240.

In certain embodiments, an output device 246 may be provided within a node 212, or accessible within the apparatus 210. Output devices 246 may include one or more physical hardware units. For example, in general, a port 244 may be used to accept inputs into and send outputs from the node 212. Nevertheless, a monitor 248 may provide outputs to a user for feedback during a process, or for assisting two-way communication between the processor 214 and a user. A printer 250, a hard drive 252, or other device may be used for outputting information as output devices 246.

Internally, a bus 254, or plurality of buses 254, may operably interconnect the processor 214, memory devices 216, input devices 224, output devices 246, network card 242, and port 244. The bus 254 may be thought of as a data carrier. As such, the bus 254 may be embodied in numerous configurations. Wire, fiber optic line, wireless electromagnetic communications by visible light, infrared, and radio frequencies may likewise be implemented as appropriate for the bus 254 and the network 240.

In general, a network 240 to which a node 212 connects may, in turn, be connected through a router 256 to another network 258. In general, nodes 212 may be on the same network 240, adjoining networks (i.e., network 240 and neighboring network 258), or may be separated by multiple routers 256 and multiple networks as individual nodes 212 on an internetwork. The individual nodes 212 may have various communication capabilities. In certain embodiments, a minimum of logical capability may be available in any node 212. For example, each node 212 may contain a processor 214 with more or less of the other components described hereinabove.

A network 240 may include one or more servers 260. Servers 260 may be used to manage, store, communicate, transfer, access, update, and the like, any practical number of files, databases, or the like for other nodes 212 on a network 240. Typically, a server 260 may be accessed by all nodes 212 on a network 240. Nevertheless, other special functions, including communications, applications, directory services, and the like, may be implemented by an individual server 260 or multiple servers 260.

In general, a node 212 may need to communicate over a network 240 with a server 260, a router 256, or other nodes 212. Similarly, a node 212 may need to communicate over another neighboring network 258 in an internetwork connection with some remote node 212. Likewise, individual components may need to communicate data with one another. A communication link may exist, in general, between any pair of devices.

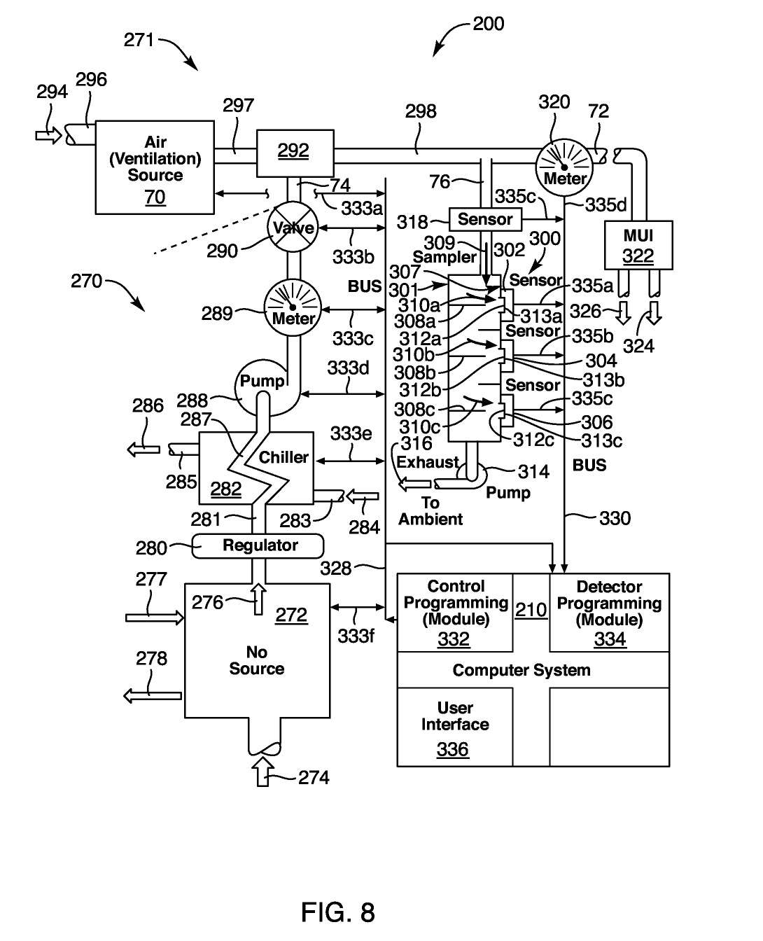

Referring to FIG. 8, a nitric oxide delivery system 200 or system 200 may rely on a computer system 210, embedded therein or otherwise operably connected thereto, in order to deliver a therapeutic gas through a gas titration system 270. Gas flows 276 including nitric oxide, other gases, or both, are measured and delivered into the flow 297 and outputs 324 of a breathable gas system 271 or air source 70 for ventilation of a subject. Therefore, a therapeutic gas titration system 270 (or simply a therapeutic gas system 270 or system 270) may interface with a breathable gas system 271. Typically, the therapeutic gas system 270 begins with a source 272 for the therapeutic gas, typically nitric oxide. The source 272 may actually provide for materials 274 input into the source 272, such as the generator 10 discussed hereinabove. In other embodiments, the source 272 may be bottled nitric oxide gas, or a pressurized tank of nitric oxide gas.

In certain embodiments, such as the generator 10 hereinabove, input materials 274 may be provided as well as other inputs 277, such as electrical power, thermal energy, other chemical constituents, other supporting materials, or the like. The result from the source 272 is an output 276 of substantially pure nitric oxide 276. Meanwhile, to the extent that materials 274 or other inputs 277 may require a discharge 278 of waste products, thermal energy rejection from thermodynamic processes, chemical processes, or the like, they may result in discharges 278.

In many embodiments, a source 272 will interface with the remainder of the therapeutic gas system 270 by a regulator 280 controlling pressure to a predetermined value for introduction into the remainder of the system 270.

In the illustrated embodiment, a line 281 may pass the therapeutic gas into a chiller 282. A chiller 282 is significant in that it has been found effective to reduce the temperature and pressure at which nitric oxide is handled. Decompression and cooling have been shown effective to reduce secondary reactions of nitric oxide into nitrogen dioxide or NO₂. Again, in the illustrated embodiment, the chiller 282 may therefore be used, particularly if the source 272 is a thermally driven generator 10.

The chiller 282 may provide an inlet 283 whereby coolant 284 is introduced in a cross-flow, counter-flow, concurrent-flow, or other arrangement in order to cool the therapeutic gas 276. The coolant 284 passing through the inlet 283 will be used to chill the therapeutic gas 276 received from the source 272. The warmed flow 286 of coolant 284 will exit through the port 285 or outlet 285 after passing over the coils 287 or passes 287. For example, good heat exchanger design may dictate more than one passage of the coils 287 through the interior of the chiller 282 for extended exposure to the coolant flow 284.

Typically, the pump 288 may be positioned downstream of the source 272, and often downstream of the chiller 282. One purpose for the pump 288 drawing on the source 272 is to maintain minimum pressures in the lines 281, 74 in order to minimize reaction of nitric oxide into nitrogen dioxide, which is considered an undesirable oxide of nitrogen.

Typically, a meter 289 or flow meter 289, illustrated schematically only, will need positioned somewhere in the line 74 feeding the therapeutic gas 276 to the breathing line 297 or flow 297. However, the position of the meter 289 and valve 290 are not necessarily critical. For example, the positions of the meter 289 and valve 290 may be switched. Likewise, the pump 288 may be positioned downstream of one or both of the meter 289 and valve 290. The pump 288 is responsible to deliver therapeutic gas 276 through the line 74 into a mixer 292 or chamber 292 that receives both the therapeutic gas and breathing air 294. The breathing air 294 may be considered an intake material through a port 296 or inlet 296 drawn into a source 70 or ventilator source 70, also simply referred to as an air source 70. This ventilator 70 is responsible to provide clean, breathable air, typically ambient air 294, and not typically pure oxygen. However, various processes may be employed to provide a flow 297 or feed 297 that will be directed to a subject (e.g., patient).

The meter 289 is best served by a float valve 289 sometimes referred to as a “pea valve” 289 that relies on a variable flow passage based on the elevation of an aerodynamically lifted indicator. This light weight indicator rests in a flow passage having a variable cross-sectional area depending on the altitude at which the float rides. The readout of the system 289 may be manual, electronic, or rely on other mechanism. The float height is a function of “pressure head” and flow rate. However, the pea valve system 289 has been found to produce precision with a minimum of obstruction, as compared with other types of metering valves 289. Thus, the flow meter 289 provides a measurement for the actual volumetric flow rate of the therapeutic gas 276 through the line 74.

The valve 290 is a metering valve. The presence of the meter 289 with the metering valve 290 is not redundant. The purpose of the meter 289 is to determine the actual volumetric flow rate of the therapeutic gas. Meanwhile, the metering valve 290 is a control element 290 that precisely controls exactly the amount of therapeutic gas flow 276 that will be permitted. More will be discussed hereinbelow regarding the metering valve 290 or control valve 290.

Ultimately, the line 74 delivers the therapeutic gas into a chamber 292 that operates as a mixer 292 with the flow 297 or line feed 297 from the ventilator 70 directed toward the subject. Thus, the flow 298 or line 298 is a mixture of the air flow 297 from the ventilator 70 and the therapeutic gas flow from the line 74 delivered from the therapeutic gas system 270.

A detector system 300 involves a series of sensors 302, 304, 306. In the illustrated embodiment, each of the sensors 302, 304, 306 operates to detect a different gas, here, nitric oxide, nitrogen dioxide, and oxygen, respectively. The sensors operate within a manifold 301 wherein each of the sensors 302, 304, 306 is mounted in or at a wall 307 of the manifold 301. Meanwhile, the operation of the sensors 302, 304, 306 and the metering by the metering valve 290 are operated in a new manner in order to obtain the precision and responsiveness required for a system 200 in accordance with the invention. For example, the metering valve 290, even when selected to be the most precise available, operating at the pressures important to the therapeutic gas delivery system 270, is wholly inadequate. That is, the precision of the best metering valves 290 available provides inadequate metering when operating in the realm of pressures (e.g., less than an atmosphere, sometimes less than a third or a fourth of that) desired for minimizing consequent reactions of the nitric oxide.

Of particular problematic nature is the backlash or tolerance that exists because the valve 290 is a threaded needle valve 290 in one currently contemplated embodiment. Necessarily, threads must have tolerances. Tolerances create slack, slop, hysteresis, or backlash. Hysteresis is the phenomenon that a movement or a change between a first state and a second state does not travel the identical path in both directions between those two states. Hysteresis is a principle understood and documented in electrical engineering and mechanical engineering literature. In the metering valve 290, hysteresis refers to the fact that movement of a needle valve in one direction is driven by engagement of respective threads on the shaft of the needle and matching, mutually engaging threads on a surrounding housing. Movement in an opposite direction requires engagement of different faces on opposite sides of the threads of the shaft and the threads of the housing. Thus, that slack or tolerance generates a mechanical hysteresis, which is excessive, in view of the precision required for a system 200 in accordance with the invention.

Likewise, the sensors 302, 304, 306 are insufficiently responsive to make measurements quickly and precisely when used in their typical manner. Each of the sensors 302, 304, 306, may operate sufficiently precisely when detecting gases in a contained vessel, tank, line, or the like operating in a steady state. For example, systems may be calibrated to account for the fact that diffusion of chemical species toward a sensor 302, 304, 306 may be accommodated as a matter of course.

Here, the sensors 302, 304, 306 are used as a feedback mechanism to control the valve 290. A rapid, transient response is needed. A combination of the diffusion gradient in a boundary layer near a face 312a, 312b, 312c of a sensor 302, 304, 306, respectively, is completely insufficient a process for sufficiently timely, accurate control. For example, typical meters 289 expect to flow an amount of nitric oxide gas on the order of about 100 parts per million in order to apply therapeutically appropriate concentrations of nitric oxide in a flow 294 or feed line 297 of breathing air 294 treated with a therapeutic gas.

It is desired, in contrast, to provide metering down to single digits of parts per million precision in the feed 298 or line 298 running to the subject. Also, it is desired to increase the concentrations up to hundreds, even thousands of parts per million in some configurations. Typically, adult concentrations may be on the order of five or six hundred parts per million and topical applications (e.g., disinfection immersion, wound immersion in nitric oxide gas flow, etc.) may involve thousands of parts per million.

Thus, in an apparatus and method in accordance with the invention, the hysteresis of the best valves 290 available coupled with the concentration gradients near the sensing faces 312 of the sensors 302, 304, 306 combine to put the needed precision completely out of reach. One should remember a reference numeral followed by trailing a letter refers to the item corresponding to the number, but the particular instance thereof corresponding to the trailing letter. Thus, we may speak of faces 312, applying to all versions or instances of the face 312, whereas the faces 312a, 312b, 312c may refer to specific instances corresponding to each of the respective sensors 302, 304, 306.

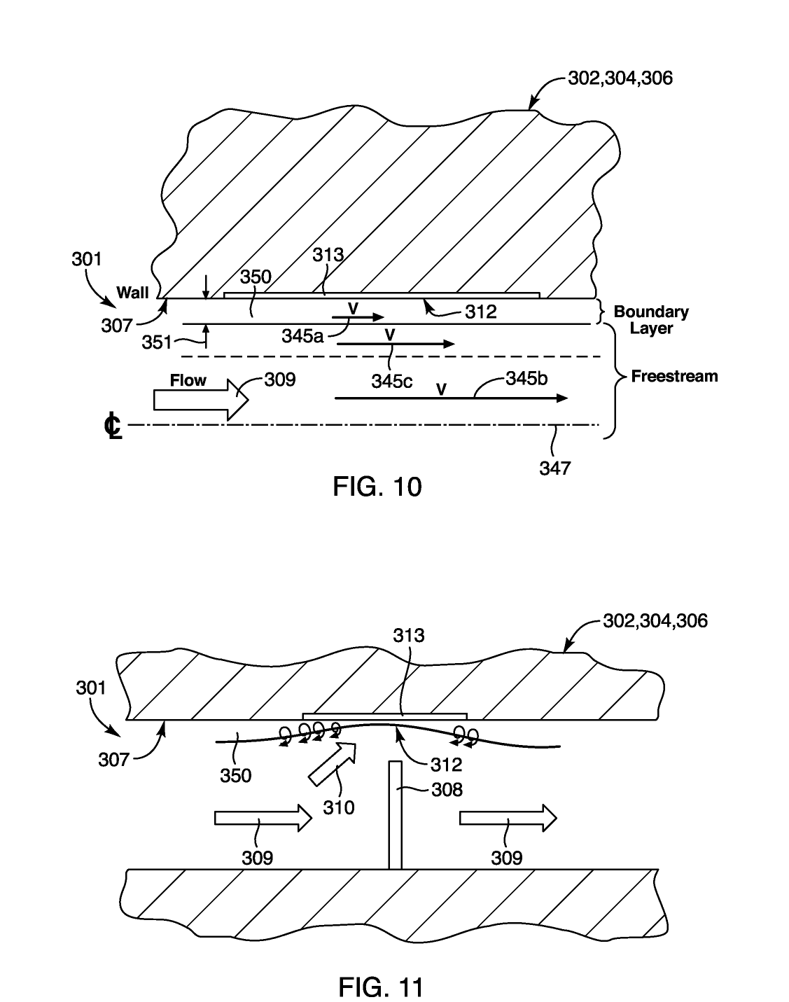

In the illustrated embodiment, the manifold 301 or chamber 301 may be constructed in a variety of configurations. However, it has been found that a mechanism is required to effectively thin or virtually destroy (reduce to some minimum value) the aerodynamic or hydrodynamic boundary layer (350, see FIGS. 10-11) against the faces 312. To that end, a series of diverters 308 divert the incoming flow 309 to a vectored flow 310 for each respective sensor 302, 304, 306. Each of the diverters 308a, 308b, 308c, corresponding to each of the vectored flows 310a, 310b, 310c, effectively strips the boundary layer 350 away from the face 312a, 312b, 312c of each of the respective sensors 302, 304, 306. More will be discussed hereinbelow regarding the operation of diverters.

However, a barrier, vane, ramp, nozzle, baffle, or other device to redirect flows 309 into vectored flows 310 provides two improvements to the performance of the sensors 302, 304, 306. First, because the boundary layer 350 is thinned, the time response for diffusion of the sensed gases approaching each of the faces 312 is dramatically reduced. The distance is reduced and the time for transport across the boundary layer 350, to the extent that any boundary layer 350 exists, is greatly reduced. Shear, mixing, and thinning all result. This improves both the accuracy, and the time response to a much better performance than would normally be expected or possible in the sensors 302, 304, 306.

Typically, the sensors 302, 304, 306 each have a sensing material 313 electronically coupled to a signal (e.g., voltage) that will be read out to a computer system 10 by the sensor 302, 304, 306. That output is a response to the presence and concentration of the specified chemical constituent being sensed. Thus, diffusion through a boundary 312 or face 312 of the sensing material 313 from the flow 310 past the face 312 is effective. With regard to the chemical process or electrochemical performance of the face 312 and material 313 for any sensor 302, 304, 306, a large barrier to diffusion is the diffusion through the boundary layer 350 within the fluid flow 309 within the manifold 301.

A pump 314 may operate upstream or downstream of the manifold 301. Regardless, the significance of the pump 314 is to draw through the line 76, a small flow 309 (comparatively speaking, with respect to flows 276, 294) from the flow 298 or line 298 that will be delivered to a subject. To that end, the pump 314 discharges an exhaust 316 overboard to the ambient. The quantity of the flow 309 is small and environmentally insignificant.

Meanwhile, one or more sensors 318 may be placed in the line 76 to detect any obstruction that may interfere with proper flow through the manifold 301. In the illustrated embodiment, the sensors 318 may include a pressure sensor, a flow meter, or the like. Thus, if the line 76 becomes occluded at any point between the feed line 298 and the pump 314, that obstruction may be timely detected and cured. Thus, the sensors 318 may include one or more sensors as deemed appropriate. A single detector of pressure has been found effective. Meanwhile, a single detector indicating flow may also serve equally well.

A meter 320 may typically be a float valve type of meter that effectively floats a comparatively light weight solid object within a vertical passage of variable cross-sectional area. Thus, with larger aerodynamic or hydrodynamic head, the float (indicator) is driven further upward against gravity. The flow, meanwhile, with increasing elevation encounters a larger cross-sectional area providing additional bypass around the indicator. This provides a non-linear response varying from a comparatively smaller flow when the float is at a lower position to a comparatively much larger flow at higher elevations of the float where the cross-sectional area is substantially increased.

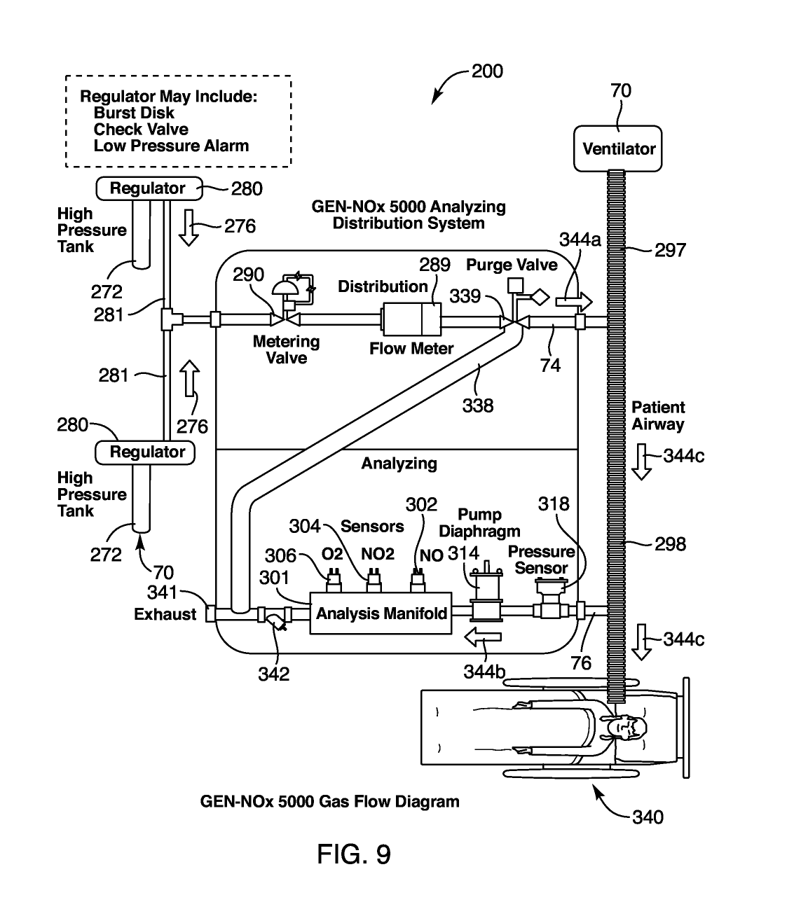

In a system 200 in accordance with the invention, a user interface 322 or mechanical user interface 322 may provide both a treatment flow 324 to a subject 340 (see FIG. 9) and an overboard discharge 326 or bypass flow 326. For example, a mechanical user interface 322 may be embodied as a breathing tent covering the upper portion of a body of an adult, an incubator tent covering a newborn infant, or the like. In other embodiments, the mechanical user interface 322 may be a mask 322, tube 322, or cannula 322 that provides a breathable gas flow 324 treated with the therapeutic gas 276 for breathing by a subject 340.

Mechanical devices such as the ventilator 70 and any driving mechanisms, such as pumps 288, fans 288, or blowers 288 associated with the ventilator 70 typically are not and cannot effectively or cost effectively be associated with the breathing process of an individual. Rather, a particular flow 72 will be delivered through the line 72 to a user interface 322 at the controlled rate. Note: any flow 297, 298, 72, may be designated by its unique line 297, 298, 72, respectively. Thus, any amount of the flow 324 used by a user will be intermittent according to a rate of breathing in and breathing out. The discharge 326 or overboard dumping 326 will accommodate the remainder of the flow 298 delivered to a subject. A tent must be vented, a cannula into the nostrils, but may be bypassed, and is, in fact, thrown overboard with each exhale by user.

In other embodiments, a mask, such as the CPAP mask or other masks delivering to mouth, nose, or both, may act as the mechanical user interface 322. The expression “mechanical” refers to the fact that this is not a data input, or even the chemical interface. Rather, the user interface 322 refers to the fact that mechanical devices move air, and direct its flow. Accordingly, a mechanical user interface 322 (e.g., mask 322, cannula 322, tube 322, CPAP 322, mouth piece 322, etc.) directs the flow 324 to a user, and accommodates the discharge overboard 326.

The therapeutic gas system 270 and the detector suite system 300 may both be operably connected to be controlled by computer system 210. In the illustrated embodiment, the ventilation source 70 may also be controlled by the computer system 210. However, this is not essential. However, it is much more valuable and much more important to control proper dosing of therapeutic gas into the flow 298. This will assure the provision of nitric oxide through the line 74 and the control of the components in the system 270 or subsystem 270. It also assures timely and accurate logging and detection from the detector suite 300.

Sensors 302, 304, 306, 318 should be precisely and timely controlled, read, and otherwise communicated with by processes executed by one or more computer 312 or processors 214 in a computer system 210 in accordance with the invention. In the illustrated embodiment, control programming 332 may be embodied as a control module 332 asserting control over the components in the therapeutic gas subsystem 270 or system 270. The inputs received from the various meters 289, 320, 333, valves 290, and sensors 302, 304, 306, 318 need to be received and processed by the detector programming 334 or detector module 334 in the computer system 210, which may be a single computer 312 or multiple, networked computer 312.

An individual operating the system 200 may set up its operation, monitor operation, and so forth including setting dosing, recording history, and the like. One may access the computer system through a user interface 336 including input systems such as a keyboard, touchscreen, number pad, mouse, and the like discussed hereinabove, as well as reading displays, monitors, alarms, and the like.

In general, a bus 328 or delivery bus 328 may include hard wiring 328, a conventional computer bus 328, or other communication link 328 permitting transmission of information to and from each of the components in the subsystem 270. Likewise, the detector suite 300 may communicate with a detector bus 330 or bus 330 providing information to the detector module responsible for processing those inputs. The delivery bus 328 may provide inputs from sensors involved with any component of the subsystem 270 to the detector module in order to process those inputs.

Command signals from the control module 332 directed to the components of the subsystem 270 may be passed along the bus 328. Any bus 328, 330 may be implemented as multiple buses 328, 330, or a single bus 328, 330, multiple wires, directly to devices, or the like. Thus, a mechanically fixed bus mechanism 328, 330 may be used, but in many environments, a more conventional computer data bus 328, 330 may serve to communicate between network aware devices or computer peripheral devices operating as components of the subsystem 270.

The connections 333 provide inputs for controlling various components as well as responses reading any detectors (e.g., 302, 304, 306, 318, etc.) provided in those components. For example, the connection 333a may communicate between the bus 328 and the ventilator 70. The communication connection 333b may pass control signals to the metering valve 290, and may report back data to the bus 328 ultimately directed to the detector module 334.

The communication link 333c or connection 333c provides information to, information from, or both, with respect to the flow meter 289. Similarly, the pump 288 may be controlled and monitored by a communication link 333d between the pump 288 and the bus 328. The chiller 282 may communicate to and from the computer system 210 over a communication link 333e. Similarly, a nitric oxide source 272 may receive control signals, report data, and the like to the computer system 210 over the bus 328 by means of a communication link 333f.

Again, each component need not have direct control or feedback control. Some systems such as a ventilator 70, may be set at a specific operating point or control position. Likewise, if a gas cylinder is used for the source 272, setting a regulator and metering valve may simply provide all the control that will be needed. However, in the illustrated embodiment, a regulator 280 as well as a metering valve 290 are both present, the latter being precisely controlled by the computer system 210.

Similarly, connections 335a, 335b, 335c communicate between the sensors 302, 304, 306, respectively, and the bus 330 serving the detector module 334. Typically, the detector module 334 may be thought of as the data acquisition module 334 responsible to pull in desired data logged from any point in the system 200, not simply the sensors 302, 304, 306.

Referring to FIG. 9, in one alternative embodiment of a system 200 in accordance with the invention, a source 272 may simply be a pressurized tank 272 equipped with a regulator 280 delivering a flow 276 or an output 276 in a set of lines 281. In the illustrated embodiment, multiple tanks 272 are illustrated, each of which may be provided with a check valve, a protection for over pressure, such as a burst disc, and likewise some indicator for approaching an empty condition, such as a low-pressure alarm. The regulators 280 regulate pressure from a tank 272 in order to provide it to the system 200 without damaging components and in order to distribute at a specific and constant rate.

In the illustrated embodiment, flows 276 through the line 281 deliver to a metering valve 290 and through a flow meter 289 a flow of the nitric oxide or other therapeutic gas. A purge valve 33 may initially divert to a purge line 338 any residual gas that is not the therapeutic gas and should be extracted from lines before operation. As a practical matter, for set up, calibration, initiation, and the like, a purge line 338 serviced by a purge valve 339 may serve to purge the line 74 of ambient air or whatever may exist within it. For example, other oxides of nitrogen may have formed from the remaining residual of nitric oxide when a system 200 was shut down.

As illustrated, the ventilator 70 provides breathing air along a line 297 representing a flow 344c or flow along a direction 344c. Meanwhile, the metering valve 290 discharges a flow 344a or a flow in a direction 344a to the line 297 for mixing. A sampling line 76 takes a small amount, not shown to scale, from the line 298, passing it by sensors 318 as described hereinabove, and driven by a pump 314, also described hereinabove. Ultimately, whether the pump 314 is upstream or downstream from the manifold 301, each of the sensors 302, 304, 306 as described hereinabove with its diverters 308 to each of the sensors 302, 304, 306 provide detection of species for analysis and feedback.

In the illustrated embodiment, the check valve 342 may assure that over pressure in the purge line 338 does not result in passing any undesired flow back into the analysis manifold 301 or from affecting the pressure and thereby changing the analysis. Ultimately, the exhaust port 341 discharges any waste from the purge line 338, as well as the flow 309 through the line 76 indicated by the direction 344b passing through the analysis manifold 301.

Ultimately, the subject 340 will receive from the line 298, through a mechanical user interface 322 the therapeutic gas 324 while any overage or overboard discharge 326 is passed to the environment.

Referring to FIG. 10, the operation of the manifold 301 in general will typically be illustrated by any particular sensor 302, 304, 306, having its boundary wall 307 containing a flow 309 therethrough. In the illustrated embodiment, the sensor face 312 on the sensor material 313 receives the species of chemical to be tested as diffused through the boundary layer 350, characterized by a thickness 351. Thus, the laminar boundary layer 350 or other boundary layer 350 will typically set up according to aerodynamic flow theory to cause a thickness 351 through which the species to be detected, measured, or “tested for” must diffuse.

Otherwise, the flow 309 has a velocity distribution illustrated by various velocities 345. The velocity 345a in the boundary layer is the slowest, and is, in fact, at a zero value at the wall 307. The velocity 345a in the boundary layer 350 varies from stationary at the wall 307, to some positive value greater than zero at the transition of the boundary layer 350 into the free stream 346. Meanwhile, the velocity 345c near but outside the edge of the boundary layer 350 is greater than the average or even maximum value of the velocity 345a in the boundary layer 350 itself. The bulk velocity 345b or maximum velocity 345b in the velocity profile within the free stream will typically be at a maximum along the center line 347 of the flow 309. The profile of all velocities 345 will be determined by the equations of fluid flow from engineering.

Referring to FIG. 11, in one aspect of an apparatus in accordance with the invention, the flow 309 is diverted by a diverter 308. The diverter 308 may be a ramp, a baffle, an obstruction, a nozzle, a channel, or any other director 308 that will redirect the flow 309 from passing through the manifold 301. The vectored flow 310 will impinge directly on the boundary layer 350 and the sensor surface 312 of the sensor material 313 in the sensors 302, 304, 306. Here, any one of the sensors 302, 304, 306 may be represented in the illustrated embodiment.

Each sensor 302, 304, 306 has a sensor material 313, and a sensor face 312 impinged upon by the vectored flow 310. The vector 310 effectively reduces to a value as close to zero as practical the thickness 351 of the boundary layer 350. Thus, the thickness 351 through which a tested species must pass, and the delay therefor has been minimized. This provides suitable speed of response, and less dependence on a steady state, calibration, and so forth. Thus, the dynamic response of the overall system 270 or subsystem 270 is greatly improved by access to more accurate, more timely, and more closely tracked concentration data for each of the tested species.

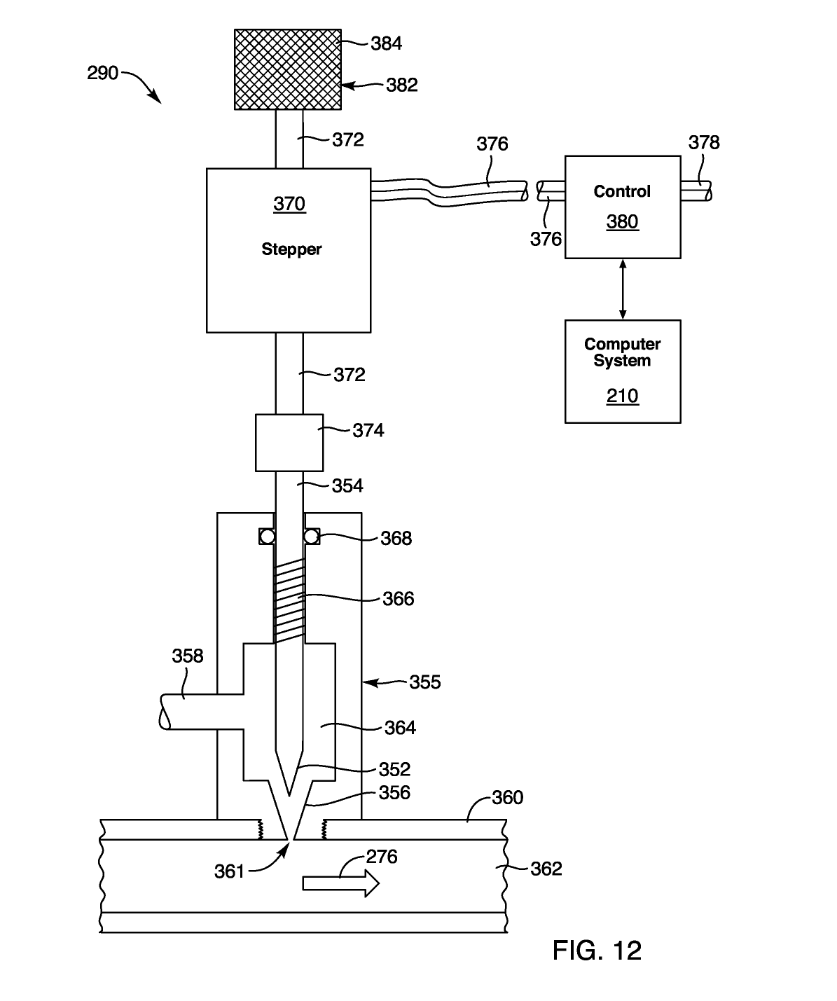

Referring to FIG. 12, in one embodiment of an apparatus and method in accordance with the invention, a metering valve 290 may be embodied as including a series of components operating to precisely meter, and to precisely control, the flow 276 of therapeutic gas through the metering therapeutic gas subsystem 270.

In the illustrated embodiment, a point 352 (needle 352) of a shaft 354 fits within a housing 355. The point 352 is machined, formed, or otherwise made to fit a seat 356 precisely. The seat 356 may be a separate component, or may be fabricated as part of the material of the housing 355. Typically, the seat 356 may be an insert 356 precisely formed and fitted into the housing 355 to receive the point 352 of the shaft 354.

Ultimately, the port 358 may receive a source of material that will be metered into the line 362 or the flow 362 within the line 360 or conduit 360. Typically, the roles or flow directions of port 358 and the line 360 may be reversed. That is, the needle valve 290 operates to simply open a metered passage between the port 358 and the line 360.

The shaft 354 is moved toward and away from the seat 356, thus providing a constriction between the point 352 of the shaft 354, and the seat 356. In the illustrated embodiment, a chamber 364 or passage 364 may be comparatively large or small, and provides for transition between the port 358 and the line 360.

Typically, threads 366 engage between the housing 355 and the shaft 354. Pitch of the threads is selected to provide several rotations of the shaft 354, each advancing the point 352 of the shaft 354 toward or away from the seat 356. Thereby, control is exercised over the passage way 361 that is the gap between the needle 352 and the seat 356. A seal 368 seals the shaft 354 to the housing 355 in order to prevent escape, and assure that all gas from the port 358 passes through the passage 361 into the conduit 360

A stepper 370 or stepper motor 370 may connect directly to a shaft 354. However, the stepper 370 with its own rotating shaft 372 is typically connected through a coupler 374 to be substantially collinear with the shaft 354. It has been found that the precision required in controlling the very best needle valves 290 available (which are manual) does not tolerate drive mechanisms. It has been found that driving is possible by a collinear alignment of the motor shaft 372 with the needle shaft 354. A coupler 374 may be of any various types, and may be a universal joint. In some embodiments it may simply be a fixed coupler 374. However, a fixed coupler 374 still tends to rigidize the alignment between the shafts 354, 372, and cause flexion which may lead to failure. Thus, various types of couplers 374 may be used including flexible couplers, universal couplers, or solid alignment shafts.

The stepper 370 or stepper motor 370 may be overridden by a knob 382 or manual interface 382 on the shaft 372. For example, knurling 284 on the knob 382, which is essentially a right circular cylinder, may operate to rotate the shaft 354 by means of manually rotating the shaft 372.

Typically, the stepper 370 is driven by power through lines 376 delivered from a controller 380 or control 380. The control 380 receives signals from a computer system 210, and power through a line 378. It then operates in accordance with the instructions from the computer system to rotate the stepper 370. The stepper motor 370 in one currently contemplated embodiment rotates a mere eight degrees on a needle valve 290 wherein the threads 366 have a pitch of less than a millimeter, thus advancing a needle valve 352 fifteen turns between fully opened (max flow) and fully closed. Thus, eight degrees out of a 360 degree circle and fifteen revolutions around that circle provide 675 increments, considerable precision in the passage 361 or control thereof.

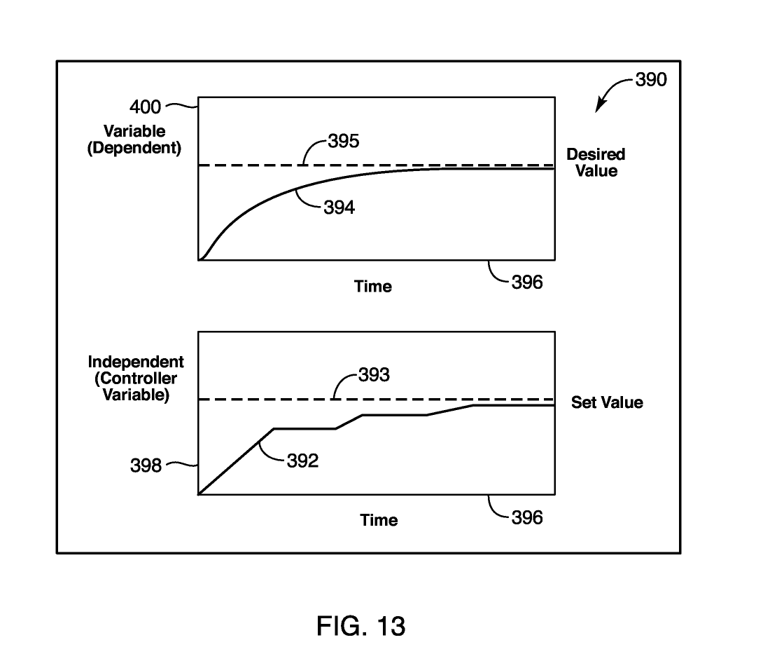

Referring to FIG. 13, the control 380 or controller 380 relies on processing by the control module 332 in the computer system 210. Lambda methods of control provide for a monotonic approach (e.g., single direction of change with no reversal) monotonically approaching a set point for the needle valve 290. This has been found to be extremely valuable and important in maintaining precision. By monotonically approaching a set point, effectively smoothly although not truly asymptotically, the precision necessary can be obtained, by avoiding any backlash.

For example, in conventional control theory, overshoot beyond a target point or set point is permitted. The control mechanism has a sufficiently high band width to draw the controlled parameter back toward the set point. For threaded needle valves and the precision of metering required here, that has not been found effective. In systems in accordance with the invention, it has been found inadequate to use conventional control theory in the available mechanical devices available today.

To control a needle valve 290 to the level of precision required by the specification of the instant invention, a chart 390 illustrates the operation of the different variables. For example, in the chart 390 or the charts 390, a curve 392 represents a value 392 of an independent variable 398. The curve 394 represents the dependent variable. For a set value 393 or set point 393 to which the needle valve 290, for example, would be positioned, the curve 392 represents the progression through a monotonic change (although shown as stepped) in that variable. Meanwhile, the set point 395 is the output or the desired dependent variable 400 that is to be set, such as flow rate.

For example, it has been found that a monotonic approach is available with no backlash using numerical methods such as predictor-corrector methods, other numerical methods that do not permit overshoot, lambda control procedures, and the like. A monotonic approach from a single side (e.g., above or below) has been shown to be extremely valuable for control of a needle valve 290 without incurring hysteresis or backlash.

In the illustrated embodiment, the curve 392 of the independent variable 398 results in a curve 394 for the dependent variable 400 that approaches a set point value 395 monotonically. Meanwhile, the time axis 396 or x axis 396 shows progress over time as the curve 394 approaches monotonically a desired value 395. Meanwhile, the value shown by the curve 392 of the independent variable 398 over a period of time 396 approaches its necessary set point 393 at whatever value obtains the result 395 for the dependent variable. That is, the set point value 393 is not really set. Rather, the value 393 becomes the point to which the curve 392 arrives by the curve 394 monotonically approaching the set point 395.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A system for precisely delivering nitric oxide, the system comprising:
a ventilator delivering breathing air;
a source of nitric oxide;
a mixer combining the nitric oxide and the breathing air into a breathing mixture;
a detector comprising a manifold receiving a portion of the breathing mixture, at least one sensor, capable of sensing at least one of oxygen, nitric oxide, and another oxide of nitrogen, and a diverter vectoring the portion of the breathing mixture toward the at least one sensor; and
the detector, wherein the diverter is sized, shaped, and positioned to reduce an aerodynamic boundary layer of the portion of the breathing mixture proximate the at least one sensor.

2. The apparatus of claim 1, further comprising:
a metering valve controlling delivery of the nitric oxide; and
a processor operably connected to the metering valve and the at least one sensor to control the delivery of the nitric oxide based on data obtained from the at least one sensor.

3. The apparatus of claim 2, wherein the processor is programmed to control the metering valve to approach a set point monotonically.

4. A method for precisely delivering nitric oxide, the system comprising:
providing a ventilator delivering breathing air;
providing a source of nitric oxide;
providing a mixer combining the nitric oxide and the breathing air into a breathing mixture;
providing a detector comprising a manifold receiving a portion of the breathing mixture, at least one sensor, capable of sensing at least one of oxygen, nitric oxide, and another oxide of nitrogen, and a diverter vectoring the portion of the breathing mixture toward the at least one sensor;
the providing the detector, wherein the diverter is sized, shaped, and positioned to reduce an aerodynamic boundary layer of the portion of the breathing mixture proximate the at least one sensor; and
providing control of a fraction of nitric oxide in the breathing mixture based on an output from the detector provided to the source of nitric oxide.

5. The method of claim 4, further comprising:
providing a metering valve controlling delivery of the nitric oxide;
providing a processor operably connected to the metering valve and the at least one sensor to control the delivery of the nitric oxide based on data obtained from the at least one sensor; and
automatically controlling the metering valve by the processor, based on the output from the detector.

6. The method of a claim 5, wherein the processor is programmed to control the metering valve to approach a set point monotonically.

7. A method of administering nitric oxide, the method comprising:
pumping a filtered gas into a reaction chamber;
providing a mixture of reactants comprising at least a calcine chromium oxide compound, a nitrite compound and a nitrate compound in the reaction chamber;
activating the reactants to initiate a reaction generating a nitric oxide gas;
evacuating the nitric oxide gas away from the reaction chamber in a closed conduit to inhibit further heating thereof and to resist further reaction of the nitric oxide gas;
filtering the nitric oxide gas; and
delivering the nitric oxide at substantially ambient conditions to a user to provide a therapeutically safe concentration of nitric oxide.

8. The method of claim 7, wherein the delivering of nitric oxide is controlled automatically by a processor based on downstream detection of the constitution of the nitric oxide mixed with air.

9. The method of claim 8, wherein a diverter improves at least one of speed and accuracy of the downstream detection by reducing a boundary layer near a sensor in the detector through vectoring a flow of the nitric oxide mixed with the air.
